(19) United States
(12) Patent Application Publication
(10) Pub. No.: US 2022/0211984 A1
(43) Pub. Date: Jul. 7, 2022

(54) Device to Reduce and Redirect Leaks

(71) Applicant: Bolanle Asiyanbola, Nashville, TN (US)

(72) Inventor: Bolanle Asiyanbola, Nashville, TN (US)

(21) Appl. No.: 17/140,051

(22) Filed: Jan. 2, 2021

(51) Int. Cl. A61M 27/00; A61B 17/11; A61F 13/36; A61F 13/511; A61F 13/00; A61F 13/537; A61F 13/513

(52) U.S. Cl. CPC A61M 27/00; A61B 17/1114; A61F 13/36; A61F 2013/530795; A61F 13/00063; A61F 13/53717; A61F 13/513; A61F 13/51104

(57) ABSTRACT

The device which may be attached inside or outside the pancreas/abdomen during surgery addresses leaks of pancreatic juice or pancreatic effluent (PE) after resection by redirecting and inactivating PE. Specifically the PE enzymes (proteases) are inactivated. Redirection is through an internal biodegradable self powered self eliminating drain with increasing gradient and then targeting or inactivating the effluent with active pharmaceutical ingredients (API) such as-protease inhibitors which are contained in graduated micro-encapsulated particles (GMEPs) coated with pH and time release shells which the device locates at optimum locations in the pancreas/abdomen. Another iteration is redirecting PE by containing the device within a mesh filled with GMEP comprising protease inhibitors (antiprotease) to inactivate PE before flowing out via openings in the mesh into the abdominal cavity as an inactivated innocuous fluid.

### FIELD OF THE INVENTION

The present invention relates to a device and method to reduce, redirect and inactivate leaks after surgery including pancreatic resection typically after removal of pancreatic tumors.

### Background—Increasing Need

Pancreatic leaks (PLs) with or without subsequent fistulas (PLSF) occur after pancreatic resections. About 90% of pancreatic resections are performed for pancreatic tumors of which pancreatic ductal adenocarcinoma; (PDAC) is the most common. The PDAC rate is increasing and projected to surpass other cancers to become the second leading cause of cancer-related deaths by 2030 [4]. PDAC is an aggressive and difficult malignancy to treat. Currently, it can only be cured through pancreatic resection. Even though various interventions used to date in the medical field have successfully reduced mortality from 60% to less than 5% today the rate of PLSFs has not concomitantly significantly changed: up to 45% of all patients, that undergo pancreatic surgery will develop PLSF] i.e. postoperative PLSFs [POPLSF] which is associated with more frequent and earlier cancer recurrence. In addition POPLSF is considered to underlie other major post operative complications (e.g., peripancreatic collections, intra-abdominal abscess, postoperative hemorrhage, sepsis, shock, and multi-organ failure), leading to prolonged in-hospital stays and increased costs.

### Background—General Medical Issues

Specifically, POPLSF are the underlying morbidity causing anastomotic dehiscence with auto-digestion of the major surgical site [MSS] i.e. anastomoses or resection sites and surrounding previously normal tissue [PNT], due to the leaking of activated pancreatic juice (APJ which is rich in enzymes (proteases).]Such leakage is known as pancreatic effluent PE because although PE consists of mainly APJ, bacteria (from the time of surgical reconstruction and intramural/intraluminal sources), jejunal secretions and sometimes bile are present in PE as well; the presence of other components in PE worsens tissue damage. PE is mainly made up of APJ which is rich in proteases, which, when activated, causes auto digestion. Despite the many alternatives that have been introduced to prevent or reduce POPLSF, none of the surgical techniques or routine care standards offers reproducible improved results including endoscopic, laparoscopic or robotic surgery or specific surgical techniques in such or adjuncts like glue or omental patching or pharmaceutical options like octreotide or analogues which function to generally reduce pancreatic ductal flow. Generally, currently, external drains are placed near the MSS. U.S. Pat. No. 8,128,682B2 (Case, Flagel, Paul) is an example of a drain and stent patent. Disadvantages of drains include passive intervention i.e. drains do not deliver any therapy to the MSS leaving the MSS to heal by itself and as such can delay initiation of chemotherapy. Drains may need to be repositioned or replaced, or they may fail, leading to reoperation or death. Protease inhibitors (PIs) have successfully been used in animal studies to treat conditions in which APJ containing proteases leaks into the abdomen causing damage. There have been scant small human trials which showed positive benefit of PIs in POPLSF. However, these studies utilized laborious methods, for example, prolonged catheter administration somewhat unfeasible in current practice of pancreatic surgery. Furthermore, these methods have no easy way of replacement/repositioning/recharging without requiring another interventional procedure. In addition, introduction of the antiprotenase is suboptimal given the limited half life and the location; which is external to the mucosa that needs to be reconstituted or reformed. Therefore, a different approach is required.

### Background—Specific Medical Issues

The head of the pancreas is on the right side of the abdomen and is connected to the duodenum (the first section of the small intestine) through a small tube called the pancreatic duct. The narrow end of the pancreas, called the tail, extends to the left side of the body. A pancreatic fistula is characterized by leakage of pancreatic fluid. Leaks into the abdomen can cause damage to previously normal tissue as well as autodigestion.

The ideal goal of any device for addressing POPLSF issues is to both inactivate the PE and redirect it to the intestine. These dual goals have not been achieved by any of the prior art. The procedure can be used for resections with or without the anastomoses being involved.

### Background—Prior Art

As mentioned earlier in Background—General Medical Issues, plastic drains and stents are the main competition. There have been other products, not related to pancreatic resections using a controlled release system useful for site specific delivery of biologically active ingredients or sensory markers, over an extended period of time. The use of biodegradable containers for drug delivery, and the use of carriers for drugs are also common. Flowable, polymer-containing compositions useful as biodegradable controlled release formulations for medicinal substances are described, for instance, in U.S. Pat. No. 4,938,763; (Dunn, English, Cowsar); U.S. Pat. No. 5,744,153 (Yewers, Kinnick, Dunn); U.S. Pat. No. 6,143,314 (Chandrashekar, Zhou, Jarr); U.S. Pat. Nos. 6,630,155; and 6,773,714 (Dunn, Garrett, Ravivarapu). One type of controlled release formulation composition includes a biodegradable, water-insoluble polymer or copolymer and medicament dissolved or dispersed in a bio-compatible organic solvent.

There also patents that use compositions comprising nanoparticles for adhering the first tissue surface to the second tissue surface in a subject that requires adhering the first tissue surface to the second tissue surface.

The device presented here has advantages as it has a shell that Is strategically fixed in the best possible position compared to the major surgical site (MSS), and it operates in two ways, first to direct PE-away from the surgical site e.g anastomoses with the use of a biodegradable drains comprising cellulose villi strands which use capillary pressure then osmotic pressure (when the cellulose portion come in contact with super absorbent particles SAP or superabsobent hydrogel[SAH]) in an increasing gradient thus providing unidirectional flow (thus constituting a drain) to pull PE into the device. The PE then comes in contact with GMEP within mini-repositories that are attached to the cellulose/SAH portion and is then inactivated. The entire device has an outer hydrophobic shell to ensure that the fluid taken up into the drain is mainly from the villi. The GMEP held within the mini-repositories consists of a biodegradable shell and active pharmaceutical ingredients API.

Principals Behind the Device

There are two parts to this device which can be used separately or together depending of the type of pancreatic resection but which are composed (i.e. release of large volume over time which is alkaline) of multiple mechanism particles in that they are not only hydrophobic but timed release and pH sensitive so that the release of pharmaceuticals better mimics the body in that only when certain multiple factors are present at certain points in time, under certain conditions will the API be released. There is a protease-antiprotenase balance that has to be maintained for optimal physiological fiction of the body. When surgery is performed and the pancreatic duct cut, pancreatic juice leaks until the surgical site heals over. When pancreatic juice, which is rich in proteases, leaks the protease-antiprotenase balance is upset with excess (digestive) proteases which interfere with wound healing thus the surgical site will not heal leading to more pancreatic juice leaking over days until the wound heals—which can take almost a year when an external drain is placed (to drain the protease elffluent/pancreatic juice away from the surgical site).

So there are three factors involved in the environment in this case—time/duration—pancreatic juice is secreted 1-1.5 L/day; proteases which are secreted in an alkaline fluid base.

Also, since the GMEP are placed at the time of surgery, there is a need for them to be able to counteract until the wound heals so timed release is important otherwise the GMEP will have to replaced potentially with another procedure. Additionally the precise calculation based on the amount of proteases secreted daily is used to calculate the amount of GMEP required.

GMEP are comprised of repository A: redirection with increasing capillary pressure to suck up pancreatic juice—with time release pH controlled GMEP surrounded by hydrophobic shells and encompassed by longitudinal strips of increasing capillary pressure Another iteration termed Repository B is comprised of mesh containing time release pH controlled GMEP sewn around/surrounding the surgical site containing API and serving as a choke point so that pancreatic juice when released from Repository B is inactivated and therefore innocuous.

How the Device Works in the Body

The method for minimizing the negative effects of PE on normal tissue is first by preventing leakage and then reducing the effects of PE on tissue GMEP consist of pH sensitive, time release enclosure which releases antiprotenase into the surgical sites. This differs from prior art in the plurality of specific combined environmental conditions which need to be met in order for the active pharmaceutical ingredient (API) to be released: need alkaline fluid of a certain amount, on a certain day to be released into the vicinity of the GMEP, thus differing from time release which deploy based on innate characteristics rather than changing environmental conditions.

Redirection is carried out via either an internal bioabsorbable drain which is self-powered using increasing gradient of capillary pressure as well as osmosis, the combination of which leads to unidirectional flow of fluid in the direction of the higher gradient thus functioning as a drain and canister which operated internally within the tissues (bowel), is bioabsorbable/eliminated with bowel movements and is self-powered even against gravity which is useful given the configuration of the reconstructed bowel where flow is against gravity at times. The increasing variant of capillary pressure is created as the wick (villi) starts the capillary pressures the first part of the drain and they They are attached to materials that are more absorbent, including super absorbent particles. These pull fluid from the villi, mainly by osmosis, because their absorptive power is greater than the capillary action i.e. increasing gradient

Furthermore, unlike prior art which are synthetic plastic drains or stents which are also placed at the time of surgery which if needed to replaced/recharged/repositioned will require another procedure, this novel drain does not. These features of the novel bioabsorbable drain are different from prior art. Furthermore this drain has internally attached GMEP which have a pH sensitive shell, which contains API of antiproteases rendering PE innocuous. The GMEP is self-eliminating with pieces attached with absorbable suture that break off when saturated and are eliminated via stool. To control the flow the outer longitudinal layer of the drain is hydrophobic.

Redirection is also carried out via an anastomosis mesh, a mesh located around the anastomosis, that is filled with time release, pH sensitive GMEP that contain antiproteases and that can sequester leaking PE, bringing such leakage into contact with antiproteases which was released from GMEP due to the pH of the PE. The overall impact of their contact is inactivation of the PE; the PE then flows out of vents in the anastomosis mesh into the general abdominal cavity as an innocuous fluid since the proteases in the PE have been inactivated.

### SUMMARY OF THE INVENTION

The device consists of redirection and inactivation. The device is placed within the bowel, close to the pancreaticojejunal anastomoses fixed by suturing or similar means consists of narrow strips of cellulose which act as villi to minimize trauma to the bowel. These strips are attached to at least one piece of a larger material which could be cellulose, which is then attached to superabsobent particle (SAP) which may be super absorbent hydrogel (SAH). This then acts in effect wicking PE from around the junction functionally acts as a unidirectional drain with capillary pressure and osmotic pressure providing the force of flow. The cellulose/SAH is attached posteriorly to the GMEP. The GMEP is comprised of time release, pH sensitive shells which contain antiproteases and SAH as the active pharmaceutical ingredient API. Other API can be antibiotics.

Once the PE flows into the SAH/antiproteases, the PE dissolves the pH controlled shell and the proteases in the PE are inactivated and the GMEP swells.

The individual pieces of the GMEP are sewn together with absorbable suture of limited half life so that they can detach and be eliminated with the stool.

There is a hydrophobic suture material that encompasses the device in place so that the main point of entry is the villi.

The second part of this method is the anastomosis mesh, named as it goes around the area surrounding the anastomosis placed around the exterior of the remnant pancreas/abdomen. This anastomosis mesh contains GMEP made of antiproteases which have timed release and pH sensitive shells. If there is an anastomotic leak, the mesh encloses the PE/PJ leak within the enclosure where the proteases of the PE will come in contact with antiproteases. The antiproteases will be released from time released, pH sensitive shells and inactivate the proteases of the PE such that innocuous fluid is released from vents in the mesh. The anastomosis mesh and the device an be used together or separately in different embodiments of the method.

It is the objective of the device to prevent and treat post-operative pancreatic leaks with or without subsequent fistuals (POPLSF) by focusing on actively healing the major surgical site (MSS) while preventing and reducing damage to the previously normal tissue (PNT) by local positioning targeted biodegradable graded microencapsulated particles (GMEPs) with active pharmaceutical ingredients in relation to the MSS, directing the effluent away from the MSS then inactivating the PE proteases

It is another objective of the device to assist in the healing at the MSS site balance between the proteases and the protease inhibitors (PI)/antiproteases. When there is a leak from the pancreas of pancreatic juice which contains proteases then imbalance occurs with more proteases than antiproteases. This has a negative effect on wound healing. By introducing PI in the GMEPs, balance can be potentially restored and this allows for better conditions for wounds to heal.

Advantages of the Device and Method Over Prior Art

| | | Internal stent | | Protease | Surgical | |
|---|---|---|---|---|---|---|
| | | or Internal/ | | Inhibitor | technique +/− | Device |
| | External | external | Octerotide/ | Systemic/ | adjuncts | Pf this |
| | Drain | stents | analogues | regional | e.g glue | application |
| Local targeted | No | No | No | No | No | Yes |
| positioning | | | | | | |
| Redirect/ | No | No | No | No | No | Yes |
| Inactivate PE | | | | | | |
| Different API | No | No | No | No | No | Yes |
| can be used | | | | | | |
| e.g. antibiotics | | | | | | |
| with poor | | | | | | |
| systemic | | | | | | |
| absorption | | | | | | |
| Self-powered | No | No | No | No | No | Yes |
| internal | | | | | | |
| device | | | | | | |
| Transfer PE | No | No | No | No | No | Yes |
| to defined | | | | | | |
| time and | | | | | | |
| place | | | | | | |
| ↓need/effect | No | No | No | Maybe | No | Yes |
| of systemic | | | | | | |
| administration | | | | | | |
| Biodegradable | No | possibly | yes | possibly | possibly | Yes |

### BRIEF DESCRIPTION OF DRAWINGS

FIG. 1 shows the pancreatic duct which exits through the connection or opening (anastomoses) into the small intestine and the placement of the device as well as the device components.

FIG. 1a, shows the device in more detail, but also highlights Repository A and Repository B.

FIG. 1b shows the inner configuration of a repository with a hydroscopic outer shell, ph sensitive biodegradable shell, and the active pharmaceutical ingredients.

FIG. 1c shows a close-up of the PDS hydrophobic outer shell and the villi that pull the PE into the device. The villi are attached to superabsorbent particles (not shown) that attach to the PDS hydrophobic outer shell.

FIG. 2 shows the anastomosis mesh, names as it is placed around the anastomosis,

FIG. 2a shows a view of half of the mesh, highlighting the GMEP with API within the mesh.

### SHORT DESCRIPTION OF THE NUMBERS WITHIN THE DRAWINGS

1. pancreatic duct.
2. anastomosis.
3. Intestine
4. villi of repository, fingerlike or threadlike projections from the surface of the device.
5. hydrophobic PDS shell.
6. anastomosis active pharmaceutical agents—super absorbent particles/antibiotic/protease inhibitors inside of anastomosis mesh.
7. pH sensitive shell that surround the active pharmaceutical agents.
8. cellulose or biodegradable material that composes the pH sensitive shell.
9. connecting suture to the pancreas wall.
10. suture for the repository pieces, separating repositories from each other
13. the anastomosis mesh with perforated openings
15. graduated micro-encapsulated particles (GMEP)
16. the repository which holds the GMEP and API.
17. active pharmaceutical ingredients (API)
18. one location of PE (pancreas effluent)
19. one location of possible leak
20. Repository A
21. Repository B
22. mesh around the mini-repositories

### DETAILED DESCRIPTION

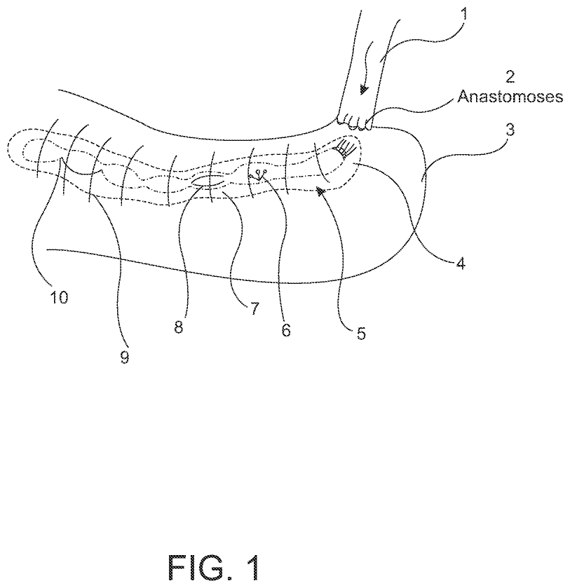

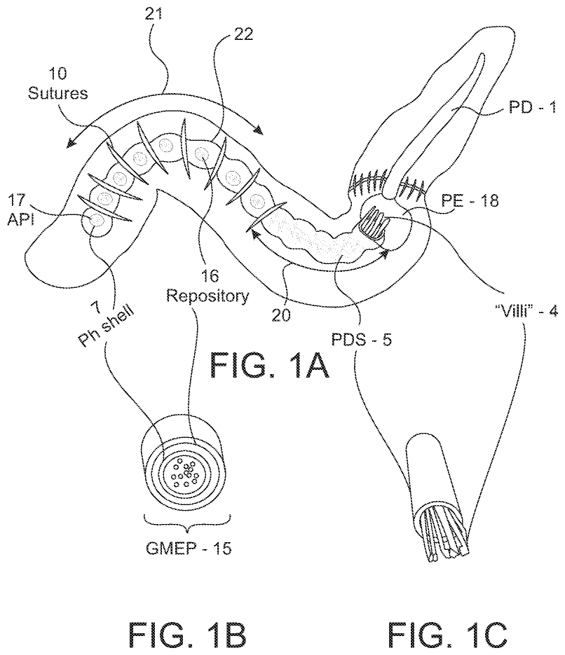

FIG. 1 shows broad over view of the device as it is placed within the body, The pancreatic duct 1 and the anastomoses lead into the intestine 3 the device villi 4, are near the connection of the pancreatic duct and intestines, pulling the PE into the device. The hydrophobic shell 5 also assists in pulling the PE away from the MSS. Active pharmaceutical ingredients 6 also may be include in GMEP is in the front section of the device, the back of the drawing shows the pH sensitive shells, 7 which are made from biodegradable material 8, the drawing also shows anchoring sutures 9 and the sutures that separate the pH sensitive shells 10.

FIG. 1a shows a more detailed look at the first embodiment of the placement of the device when it is placed into the inside of the intestine. The device is held in place by connecting sutures to the bowel wall 10. The device is placed after the pancreatic duct 1, anastomoses 2 in the intestine adjacent to the anastomoses such that the pancreatic effluent steam connects with the device. Since the device has “villi” 4 (—small protrusions from the device), the villi 4 are the part of the device that interact with the steam of pancreatic effluent directly in the front section of the device, Repository A, 20. The hydrophobic PDS shell) allows PE to be drawn through the shell into an area that can hold effluent flow 16 so the PE can interact with the active pharmaceutical agents 17. The active pharmaceutical agents 17 are within graduated micro encapsulated particles 15, that are held in repositories within the inside of a series pH sensitive shells 7, Repository B, 21, the shells are composed on biodegradable material of varying thicknesses for each shell to allow the active pharmaceutical agents 17 to be released at varying times. The series of pH sensitive shells 7 that are separated by anchoring sutures 10, which attach to the outer mesh (22)

FIG. 1a shows the inside of a repository, the hydrophobic PDS outer shell 5, the area for effluent flow inside of repository 16, the inner pH biodegradable shell 7, the graduated micro-encapsulated particles (GMEP)15 that contain the active pharmaceutical ingredients 17.

FIG. 1b shows the opening of the hydrophobic PDS shell 5 with the villi 4, which together such the PE effluent down towards the repositories. The villi 4 are attached to superabsorbent particles that attach to the all of the hydrophobic PDS shell 5 (not shown).

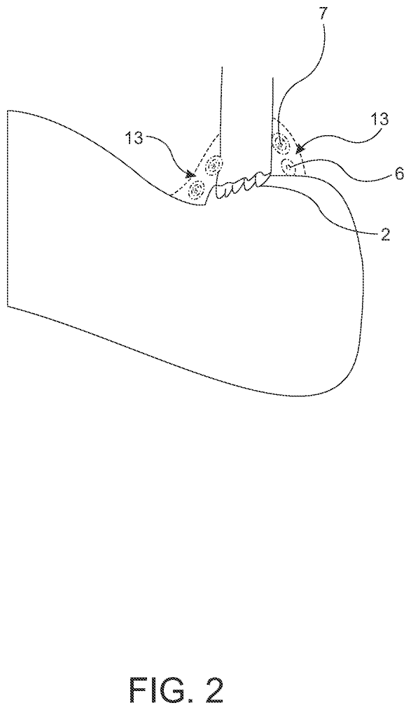

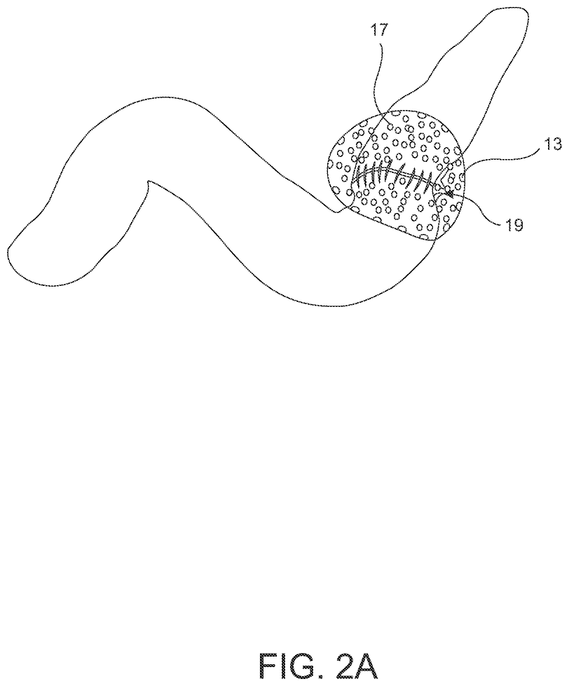

FIG. 2 shows the anastomosis mesh with perforated openings 13, which is placed where there are likely leaks 19, and also the active pharmaceutical ingredient within the mesh 19.

FIG. 2a shows a view of half of the anastomosis mesh 13, highlighting API 6 inside of the pH sensitive shells 7 within the anastomosis mesh 13.

1. A method to reduce, redirect and inactivate pancreatic leaks after pancreatic sections comprising:
Use of a material around within or outside the anastomosis area which contains graduated micro encapsulated particles with pH sensitive biodegradable shells containing active pharmaceutical ingredients;
a device to reduce and redirect leaks after surgery comprising:
a hydrophobic permeable shell at one end of the device acting as a drain that is placed close to a source of leaks of pancreatic effluent;
villi, small “finger like” protrusions from the outer end of the hydrophobic permeable shell acting as a drain that attaches to super absorbent particles that attach to the wall of the hydrophobic permeable shell;
a series of cellulose repositories on the other end of the device attached to time release, pH sensitive biodegradable shells that contain active pharmaceutical agents including at least one of these or a combination of antproteases, super absorbent particles, antibiotics that degrades based on the alkaline level of the pancreatic fluid;
the cellulose repositories containing an open area for receiving PE and graduated micro-encapsulated particles containing active pharmaceutical ingredients;
the repositories separated by anchoring sutures which attach to the outer hydrophobic shell;
whereby when the device is positioned for in areas where leakage is expected and the device villi and the hydrophobic permeable shell attracts leakage through the device to the end where it can absorbed or treated by the active pharmaceutical ingredients in the cellulose depositories as those ingredients are released by the graduated microencapsulated particles;
whereby the device is self-powered by means of internal intermolecular forces, diffusion gradients and osmosis;
whereby the device is biodegradable;
whereby the device is self-eliminating with bowel movements.

2. The method of claim 1 where the method is used without the material around within or outside of the anastomosis area.

3. The method of claim 1 where the method is used without the device.

4. The device in the method of claim 1 where the hydrophobic permeable part at one end of the device may also include, graduated micro-encapsulated particles containing active pharmaceutical ingredients.

5. The method of claim 1 where wall thicknesses of the pH sensitive biodegradable covering vary to allow different time releases points for each repositories.

6. The device method of claim 1 where the inner shells are not pH sensitive, but instead enzyme sensitive, ion sensitive or have another sensitivity to activate the biodegradable process,

7. The device in the method of claim 1 where the mini-repositories and inner pH sensitive shells are not attached inner lining of the intestine.

8. The device in the method of claim 1 where redirection is increased with the increased use or different properties of in the hydrophobic shell of desiccants, super absorbent particles, polymers, hydrogel, cellulose derivatives or other absorbent material.

9. The device in the method of claim 1 where redirection is varied with varying properties of the hydrophobic shell of desiccants, super absorbent particles, polymers, hydrogel, cellulose derivatives or other absorbent material.

10. The device in the method of claim 1 where inactivating the PE can be increased with particles in the repositories such as protease inhibitors, water saline solutions and other materials that can inactivate or dilute leaks.

11. The device in the method of claim 1 where inactivating the PE can be varied with varying particles in the repositories such as protease inhibitors, water saline solutions and other materials that can inactivate or dilute leaks.

12. The device in the method of claim 1 where wound healing can be varied due to varying particles in the repositories such as antibiotics, protease inhibitors, antibiotics, water saline solutions and other materials that can vary wound healing.

13. The drain section of device in the method of claim 1 consisting of the hydrophobic permeable shell and the villi for use in other applications.
